SEQUENCE LISTING (2 sequences)

SEQ ID NO: 1 - protein, 510 aa, Homo sapiens
  SIGNAL (1)..(20)
  mat_peptide (21)..(510)
MRPGLSFLLALLFFLGQAAGDLGDVGPPIPSPGFSSFPGVDSSSSFSSSSRSGSSSSRSLGSGGSVSQLFSNFTGSVDDRGTCQCSVSLPDTTFPVDRVERLEFTAHVLSQKFEKELSKVREYVQLISVYEKKLLNLTVRIDIMEKDTISYTELDFELIKVEVKEMEKLVIQLKESFGGSSEIVDQLEVEIRNMTLLVEKLETLDKNNVLAIRREIVALKTKLKECEASKDQNTPVVHPPPTPGSCGHGGVVNISKPSVVQLNWRGFSYLYGAWGRDYSPQHPNKGLYWVAPLNTDGRLLEYYRLYNTLDDLLLYINARELRITYGQGSGTAVYNNNMYVNMYNTGNIARVNLTTNTIAVTQTLPNAAYNNRFSYANVAWQDIDFAVDENGLWVIYSTEASTGNMVISKLNDTTLQVLNTWYTKQYKPSASNAFMVCGVLYATRTMNTRTEEIFYYYDTNTGKEGKLDIVMHKMQEKVQSINYNPFDQKLYVYNDGYLLNYDLSVLQKPQ

SEQ ID NO: 2 - protein, 452 aa, Artificial Sequence
  Recombinant Peptide
GSGGSVSQLFSNFTGSVDDRGTCQCSVSLPDTTFPVDRVERLEFTAHVLSQKFEKELSKVREYVQLISVYEKKLLNLTVRIDIMEKDTISYTELDFELIKVEVKEMEKLVIQLKESFGGSSEIVDQLEVEIRNMTLLVEKLETLDKNNVLAIRREIVALKTKLKECEASKDQNTPVVHPPPTPGSCGHGGVVNISKPSVVQLNWRGFSYLYGAWGRDYSPQHPNKGLYVAPLNTDGRLLEYYRLYNTLDDLLLYINARELRTYGQGSGTAVYNNNMYVNMYNTGNIARVNLTTNTIAVTQTLPNAAYNNRFSYAVAWQDIDFAVDENGLWVIYSTEASTGNMVISKLNDTTLQVLNTWYTKQYKPSASNAFMVCGVLYATRTMNTRTEEIFYYDTNTGKEGKLDIVMHKMQEKVQSINYNPFDQKLYVYNDGYLLNYDLSVLQKPQHHHHHH

(19) United States
(12) Patent Application Publication
(10) Pub. No.: US 2012/0276561 A1
(43) Pub. Date: Nov. 1, 2012

(54) MARKER PROTEIN FOR TYPE-2 DIABETES

(75) Inventors: Laura Badi, Basel (CH); Martin Ebeling, Grenzach-Wyhlen (DE); Hugues Matile, Basel (CH); Cristiano Migliorini, Genf (CH); Jacob Sabates Bellver, Zuerich (CH); Thomas Schindler, Loerrach (DE); Elena Sebokova, Basel (CH); Haiyan Wang, Allischwil (CH)

(73) Assignee: Hoffmann-La Roche Inc., Nutley, NJ (US)

(21) Appl. No.: 13/512,102

(22) PCT Filed: Nov. 23, 2010
(86) PCT No.: PCT/EP2010/067963

§ 371 Date: May 25, 2012

(30) Foreign Application Priority Data
Nov. 26, 2009 (EP) 09177238.4

(51) Int. Cl. G01N 33/577; C07K 16/18

(52) U.S. Cl. 435/7.92; 530/388.2

(57) ABSTRACT

The present invention provides a marker protein for the early detection of type II diabetes, antibodies directed to the marker protein and their use in a diagnostic method for type II diabetes and in drug development.

The present invention provides a diagnostic marker protein for the early detection of type II diabetes, antibodies directed to the marker protein and their use in a diagnostic method for type II diabetes and in drug development.

Type 2 diabetes (non-insulin dependent diabetes mellitus (NIDDM)) is a disorder that is characterized by high blood glucose in the context of insulin resistance and relative insulin deficiency. There are an estimated 23.6 million people in the U.S. (7.8% of the population) with diabetes with 17.9 million being diagnosed, 90% of whom are type 2. With prevalence rates doubling between 1990 and 2005, CDC (Centers for Disease Control and Prevention) has characterized the increase as an epidemic.

Therefore, there is a need for diagnostic markers and methods allowing an early detection of type II diabetes.

In a first object the present invention relates to a method for diagnosis of type II diabetes or for determining the predisposition of an individual for developing type II diabetes comprising the steps of:

measuring in a tissue sample of the individual a level of Olfactomedin 4 (OLFM4) polypeptide, wherein a decreased level of OLFM4 polypeptide in the sample of the individual compared to a level of OLFM4 polypeptide representative for a healthy population is indicative for type II diabetes or a predisposition for developing type II diabetes.

In a preferred embodiment, the tissue is blood, preferably plasma.

In a second object, the present invention provides a method for the identification of a compound for the treatment of type II diabetes comprising the steps of:
a) administering the compound to a non-human animal suffering from type II diabetes,
b) measuring in a tissue sample of the non-human animal of step a) a level of OLFM4 polypeptide, wherein an altered level of OLFM4 polypeptide in the tissue sample of the non-human animal of step a) compared to the level of OLFM4 polypeptide in a tissue sample of an non-human animal suffering from type II diabetes to which no compound has been administered is indicative for a compound for the treatment of type II diabetes.

In a preferred embodiment, the tissue sample is blood, preferably plasma.

In a further preferred embodiment, the non-human animal is a rodent, preferably a mouse or rat, more preferably a DIO mouse, an ob/ob mouse or a ZDF rat.

In a third object, the present invention relates to a use of a OLFM4 polypeptide for the diagnosis of type II diabetes or for determining a predisposition of an individual for developing type II diabetes.

In a preferred embodiment, the OLFM4 polypeptide is the human OLFM4 polypeptide. The amino acid sequence of human OLFM4 is disclosed in Seq. Id. No. 1.

In a fourth object, the present invention provides a use of an antibody specifically binding to an OLFM4 polypeptide for the diagnosis of type II diabetes or for determining a predisposition of an individual for developing type II diabetes.

In a preferred embodiment, the antibody binds to human OLFM4 polypeptide.

In a fifth object, the present invention relates to a kit for the diagnosis of type II diabetes or determining the predisposition for developing type II diabetes in an individual comprising:
a) an antibody specific for an OLFM4 polypeptide, preferably an antibody of the present invention,
b) a labeled antibody binding to OLFM4 captured by the antibody of a) or a labeled antibody binding to the antibody of a) and
c) reagents for performing a diagnostic assay.

In a preferred embodiment, the specific antibody for the OLFM4 polypeptide binds the human OLFM4 polypeptide.

The methods of the present invention can be used to monitor type II diabetes therapy response in patients undergoing diabetes therapy by measuring the level of OLFM4 polypeptide in tissue samples of these patients, preferably in blood samples. Patients showing an altered level of OLFM4 polypeptide in a tissue sample in the course of therapy compared to the OLFM4 polypeptide level at the beginning of the therapy respond to the diabetes therapy.

In a further object the present invention relates to a monoclonal antibody directed to human OLFM4 polypeptide.

In a preferred embodiment, the antibody is an antibody comprising a CDR1 to CDR3 of a V_{H} domain of an antibody obtainable from a hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010) and a CDR1 to CDR3 of a V_{L} domain of an antibody obtainable from a hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010).

In a further preferred embodiment, the antibody is a chimeric antibody comprising a V_{H} domain and a V_{L} domain of an antibody obtainable from the hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010).

In a further preferred embodiment, the antibody is produced by the hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010).

Methods for detection and/or measurement of polypeptides in biological samples are well known in the art and include, but are not limited to, Western-blotting, ELISAs or RIAs, or various proteomics techniques. Monoclonal or polyclonal antibodies recognizing the OLFM4 polypeptide/fragments thereof, or peptide fragments thereof, can either be generated for the purpose of detecting the polypeptides or peptide fragments, e.g. by immunizing rabbits with purified proteins, or known antibodies recognizing the polypeptides or peptide fragments can be used. For example, an antibody capable of binding to the denatured proteins, such as a polyclonal antibody, can be used to detect OLFM4 polypeptide/fragments thereof in a Western Blot. An example for a method to measure a marker is an ELISA. This type of protein quantitation is based on an antibody capable of capturing a specific antigen, and a second antibody capable of detecting the captured antigen. Methods for preparation and use of antibodies, and the assays mentioned hereinbefore are described in Harlow, E. and Lane, D. Antibodies: A Laboratory Manual, (1988), Cold Spring Harbor Laboratory Press.

In a further object the present invention provides a method for the detection of pancreatic β-cells in a tissue sample comprising:
a) providing a pancreatic tissue sample of an individual or a non-human animal,
b) detecting OLFM4 positive cells in the tissue sample of a), wherein the OLFM4 positive cells are β-cells.

In a preferred embodiment, the OLFM4 positive cells are detected by an antibody specific for OLFM4, preferably an antibody of the present invention.

The method for the detection of β-cells in a tissue sample of a human invidvidual or a non-human animal can be used for assessing the effect of type II diabetes therapy on the physiology/histology of the pancreas. For example, during the development of a compound for the treatment of type II diabetes, the method for the detection of β-cells of the present invention can be used to assess whether the compound has an effect on the physiology/histology of the pancreas i.e. whether the compound can reverse some of the effects of type II diabetes on the pancreas in a animal model for type II diabetes.

In a further object, the present invention provides a kit for the detection of β-cells in a pancreas tissue sample comprising:
a) an antibody specific for an OLFM4 polypeptide, preferably an antibody of the present invention,
b) a labeled antibody binding the antibody of a) or a labeled antibody specific for a OLFM4 polypeptide and
c) reagents for performing an immunohistochemistry assay.

Synonyms for the polypeptide Olfactomedin 4 (OLFM4) are hGC-1 and GW112.

The term “polypeptide” as used herein, refers to a polymer of amino acids, and not to a specific length. Thus, peptides, oligopeptides and protein fragments are included within the definition of polypeptide.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

The term “antibody” encompasses the various forms of antibody structures including but not being limited to whole antibodies and antibody fragments. The antibody according to the invention is preferably a humanized antibody, chimeric antibody, or further genetically engineered antibody as long as the characteristic properties according to the invention are retained.

“Antibody fragments” comprise a portion of a full length antibody, preferably the variable domain thereof, or at least the antigen binding site thereof. Examples of antibody fragments include diabodies, single-chain antibody molecules, and multispecific antibodies formed from antibody fragments. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain binding to ANG-2, namely being able to assemble together with a VH domain to a functional antigen binding site and thereby providing the property

The terms “monoclonal antibody” or “monoclonal antibody composition” as used herein refer to a preparation of antibody molecules of a single amino acid composition.

The term “chimeric antibody” refers to an antibody comprising a variable region, i.e., binding region, from one source or species and at least a portion of a constant region derived from a different source or species, usually prepared by recombinant DNA techniques. Chimeric antibodies comprising a murine variable region and a human constant region are preferred. Other preferred forms of “chimeric antibodies” encompassed by the present invention are those in which the constant region has been modified or changed from that of the original antibody to generate the properties according to the invention, especially in regard to Clq binding and/or Fc receptor (FcR) binding. Such chimeric antibodies are also referred to as “class-switched antibodies.”. Chimeric antibodies are the product of expressed immunoglobulin genes comprising DNA segments encoding immunoglobulin variable regions and DNA segments encoding immunoglobulin constant regions. Methods for producing chimeric antibodies involve conventional recombinant DNA and gene transfection techniques are well known in the art. See e.g. Morrison, S. L., et al., Proc. Natl. Acad. Sci. USA 81 (1984) 6851-6855; U.S. Pat. Nos. 5,202,238 and 5,204,244.

The term “human antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 33-40). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). As already mentioned for chimeric and humanized antibodies according to the invention the term “human antibody” as used herein also comprises such antibodies which are modified in the constant region to generate the properties according to the invention, especially in regard to Clq binding and/or FcR binding, e.g. by “class switching” i.e. change or mutation of Fc parts (e.g. from IgG1 to IgG4 and/or IgGl/IgG4 mutation.).

The term “epitope” includes any polypeptide determinant capable of specific binding to an antibody. In certain embodiments, epitope determinant include chemically active surface groupings of molecules such as amino acids, sugar side chains, phosphoryl, or sulfonyl, and, in certain embodiments, may have specific three dimensional structural characteristics, and or specific charge characteristics. An epitope is a region of an antigen that is bound by an antibody.

The “variable domain” (variable domain of a light chain (V_{L}), variable domain of a heavy chain (V_{H})) as used herein denotes each of the pair of light and heavy chain domains which are involved directly in binding the antibody to the antigen. The variable light and heavy chain domains have the same general structure and each domain comprises four framework (FR) regions whose sequences are widely conserved, connected by three “hypervariable regions” (or complementary determining regions, CDRs). The framework regions adopt a β-sheet conformation and the CDRs may form loops connecting the β-sheet structure. The CDRs in each chain are held in their three-dimensional structure by the framework regions and form together with the CDRs from the other chain the antigen binding site. The antibody's heavy and light chain CDR3 regions play a particularly important role in the binding specificity/affinity of the antibodies according to the invention and therefore provide a further object of the invention.

The term “antigen-binding portion of an antibody” when used herein refer to the amino acid residues of an antibody which are responsible for antigen-binding. The antigen-binding portion of an antibody comprises amino acid residues from the “complementary determining regions” or “CDRs”. “Framework” or “FR” regions are those variable domain regions other than the hypervariable region residues as herein defined. Therefore, the light and heavy chain variable domains of an antibody comprise from N- to C-terminus the domains FR1, CDR1, FR2, CDR2, FR3, CDR3, and FR4. Especially, CDR3 of the heavy chain is the region which contributes most to antigen binding and defines the antibody's properties. CDR and FR regions are determined according to the standard definition of Kabat et al., Sequences of Proteins of Immunological Interest, 5th ed., Public Health Service, National Institutes of Health, Bethesda, Md. (1991) and/or those residues from a “hypervariable loop”.

Monoclonal antibodies may be prepared using hybridoma methods, such as those described by Kohler and Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent.

Any of the many well known protocols used for fusing lymphocytes and immortalized cell lines can be applied for the purpose of generating a monoclonal antibody of the present invention (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind, e.g., using a standard ELISA assay.

Antibodies may be produced using recombinant methods and compositions, e.g., as described in U.S. Pat. No. 4,816,567. In one embodiment, isolated nucleic acid encoding an anti-OLFM4 antibody described herein is provided. Such nucleic acid may encode an amino acid sequence comprising the V_{L} and/or an amino acid sequence comprising the V_{H} of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-TMEM27 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody of the present invention, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.). After expression, the antibody may be isolated from the bacterial cell paste in a soluble fraction and can be further purified.

Methods to clone antibody genes from hybridoma cells producing monoclonal antibodies are know to a person skilled in the art. For example, the genetic information for the variable heavy and light chain domains (V_{H} and V_{L}) can be amplified from hybridoma cells using polymerase chain reaction (PCR) with immunoglobulin-specific primers (Methods Mol Med. 2004;94:447-58). The nucleic acid encoding the variable heavy and light chain domains (V_{H} and V_{L}) can then be cloned in a suitable vector for expression in host cells.

### SHORT DESCRIPTION OF THE FIGURES

FIG. 1A: Detection of OLFM4 polypeptide in 10 human plasma samples by ELISA using the antibody pair OLFM4-1/23 and OLFM4-2/3,

FIG. 1B: Detection of OLFM4 polypeptide in 10 human plasma samples by ELISA using the antibody pair OLFM4-2/1 and OLFM4-2/28,

FIG. 1C: Detection of OLFM4 polypeptide in 10 human plasma samples by ELISA using the antibody pair OLFM4-2/28 and OLFM4-2/14,

FIG. 2A: Immunoprecipitation (IP) with 10 human plasma samples using the monoclonal antibody OLFM4-2/1,

FIG. 2B: Immunoprecipitation (IP) with 10 human plasma samples using the monoclonal antibody OLFM4-2/3,

FIG. 2C: Immunoprecipitation (IP) with 10 human plasma samples using the monoclonal antibody OLFM4-2/28,

FIG. 2D: Immunoprecipitation (IP) with 10 human plasma samples using the monoclonal antibody OLFM4-2/14,

FIG. 3A: Detection of OLFM4 polypeptide in plasma samples of human subjects selected from the groups: Healthy controls, Impaired Fasting Glucose (IFG), Impaired Glucose Tolerance (IGT), Impaired Fasting Glucose+Impaired Glucose Tolerance (IFG+IGT), Type 1 diabetes patients (T1D) and Type 2 diabetes patients (T2D) by ELISA using the antibody pair OLFM4 2/1 and OLFM4 2/28,

FIG. 3B: Detection of OLFM4 polypeptide in plasma samples of human subjects selected from the groups: Healthy controls, Impaired Fasting Glucose (IFG), Impaired Glucose Tolerance (IGT) and Impaired Fasting Glucose+Impaired Glucose Tolerance (IFG+IGT), Type 1 diabetes patients (T1D) and Type 2 diabetes patients (T2D) by ELISA using the antibody pair OLFM4 2/28 and OLFM4 2/14,

FIG. 4A: Immunohistochemistry (IHC) staining of Human Tissue Array using the monoclonal antibody hOLFM4 1/46,

FIGS. 4B and C: Human pancreatic islets stained with monoclonal antibody hOLFM4 1/46 (OLFM4: green, glucagon: red, DAPI: blue).

### EXAMPLES

Monoclonal Anti Human OLFM4 Antibodies of the Present Invention

The following five mouse hybridoma cell lines producing monoclonal antibodies against human OLFM4 have been deposited with the DSMZ—(Deutsche Sammlung von Mikroorganismen and Zellkulturen GmbH) on Oct. 7, 2009 in the name of F. Hoffmann-La Roche Ltd. and received the below listed deposit numbers:
OLMF4-1/23=DSM ACC3010
OLMF4-1/46=DSM ACC3011
OLMF4-2/3=DSM ACC3012
OLMF4-2/1=DSM ACC3013
OLMF4-2/14=DSM ACC3014
OLMF4-2/28=DSM ACC3015

Generation of Mouse Monoclonal Antibodies Against Human OLFM4 (Mouse OLFM4 mAbs)

The amino acid sequence of the recombinant human OLFM4 fusion polypeptide used for producing monoclonal antibodies is given below:

(Seq. Id. No. 2)
GSGGSVSQLFSNFTGSVDDRGTCQCSVSLPDTTFPVDRVERLEFTAHVLS
QKFEKELSKVREYVQLISVYEKKLLNLTVRIDIMEKDTISYTELDFELIK
VEVKEMEKLVIQLKESFGGSSEIVDQLEVEIRNMTLLVEKLETLDKNNVL
AIRREIVALKTKLKECEASKDQNTPVVHPPPTPGSCGHGGVVNISKPSVV
QLNWRGFSYLYGAWGRDYSPQHPNKGLYVAPLNTDGRLLEYYRLYNTLDD
LLLYINARELRTYGQGSGTAVYNNNMYVNMYNTGNIARVNLTTNTIAVTQ
TLPNAAYNNRFSYAVAWQDIDFAVDENGLWVIYSTEASTGNMVISKLNDT
TLQVLNTWYTKQYKPSASNAFMVCGVLYATRTMNTRTEEIFYYDTNTGKE
GKLDIVMHKMQEKVQSINYNPFDQLYVYNDGYLL-NYDLSVLQKPQHHHH
HH

Mice immunized with 5m/injection with recombinant OLFM4, produced in insect cells, coupled to a His tag. Immunizations on day 0, 13 and 28 in ImmunEasy adjuvant (ALHY-DROGEL 2%+CPG-ODN) ip in a volume of 20 μl. Evaluation of the immune response of the animals by ELISA on the recombinant OLFM4 with bleedings from day 41.Superboost at day 56 (5 μg recombinant OLFM4 in PBS iv) of 2 selected animals and fusion of the spleen cells with PAI-cells 2 days later. Hybridoma screening as well as cloning evaluation performed by ELISA on the recombinant OLFM4.

ELISA Specificity Verification

Three pairs of mAbs of the present invention were used in ELISA:

| | coating-mAb | detection-mAb |
|---|---|---|
| 1 | OLFM4-1/23 | OLFM4-2/3-Biotin |
| 2 | OLFM4-2/1 | OLFM4-2/28-Biotin |
| 3 | OLFM4-2/28 | OLFM4-2/14-Biotin |

ELISA-Results of 10 Control Human Plasmas

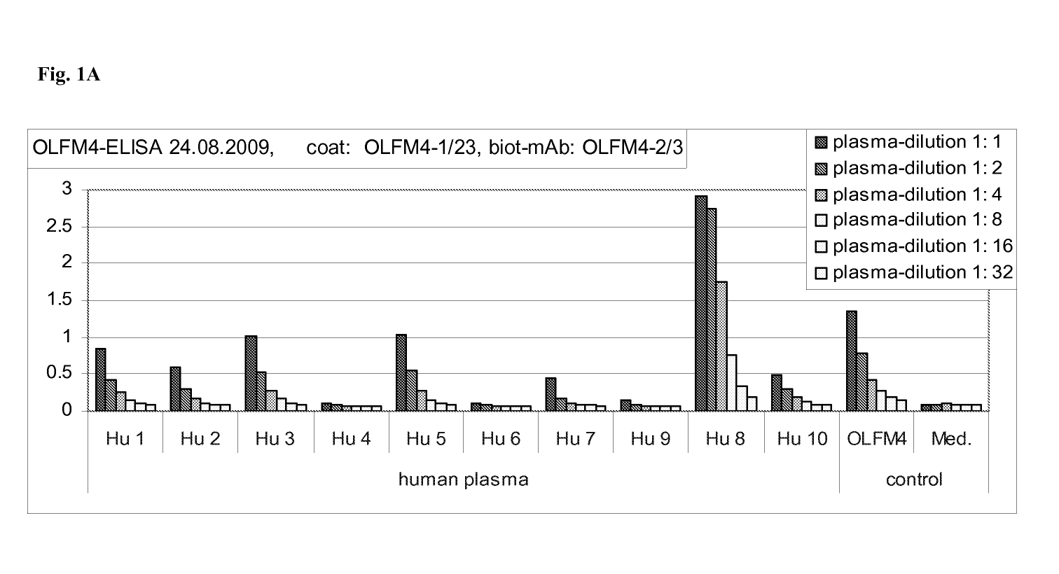

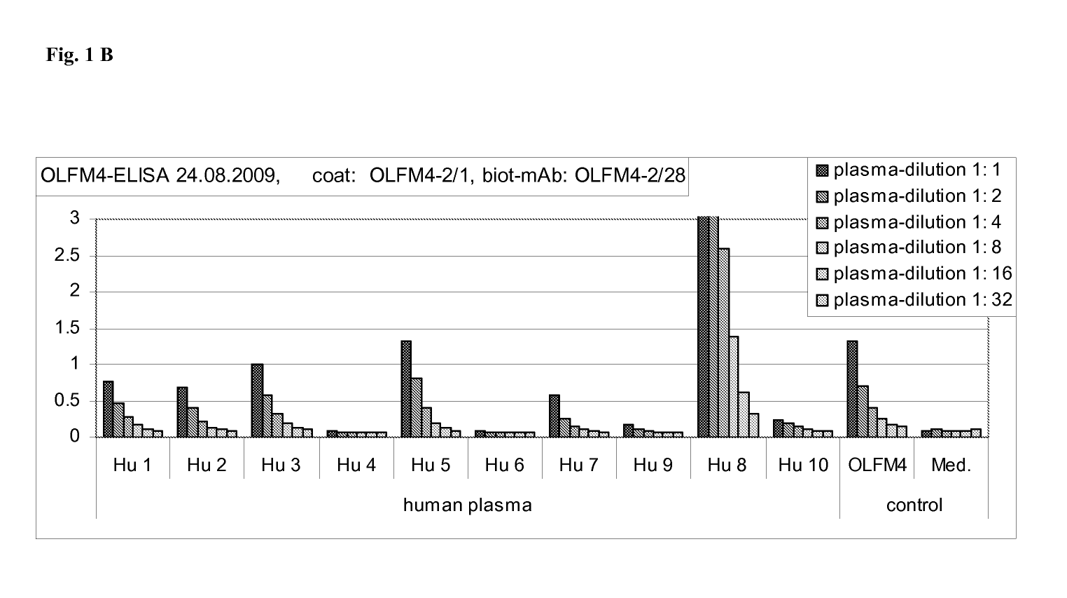

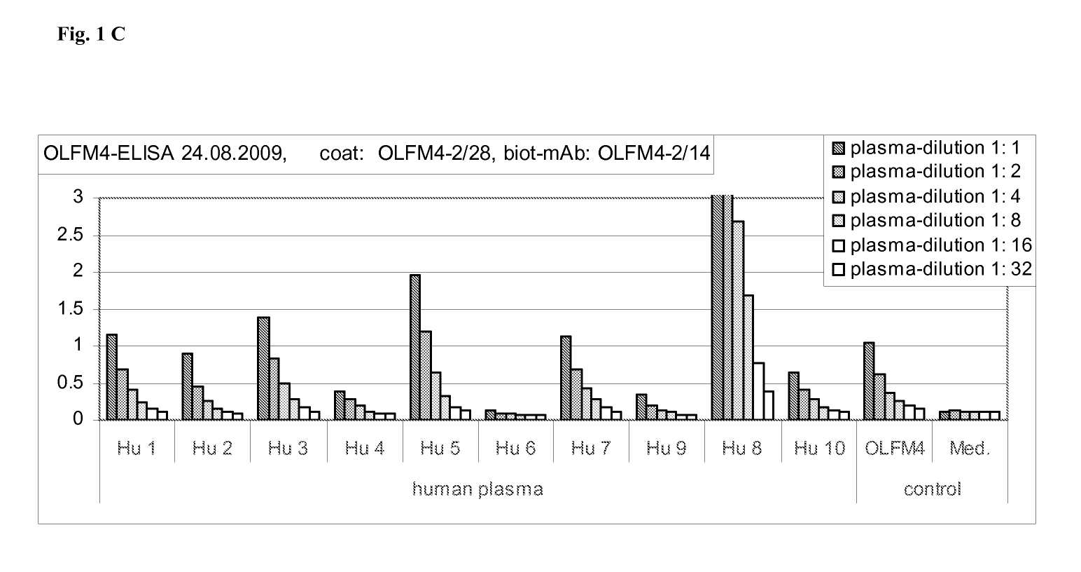

The results of the assays are given in FIG. 1A-FIG. 1C:

Hu 1-Hu 10: control human sera (blood donor human plasma)

Positive control (OLFM4): INS-1 hOLFM4 WT F11

Negative control (med): Medium

The use of three different ELISA assays to test 10 human plasma samples gave very similar results. This verified the assay specificity. These samples were further used to qualitatively verify these ELISA results by immunoprecipitation (IP).

Qualitative Validation of ELISAs by IP (Immune Rrecipitation (IP)—Final Selection of mAb Pairs: Blood Donor Human Plasma and INS-1 hOLFM4 WT F11/Medium)

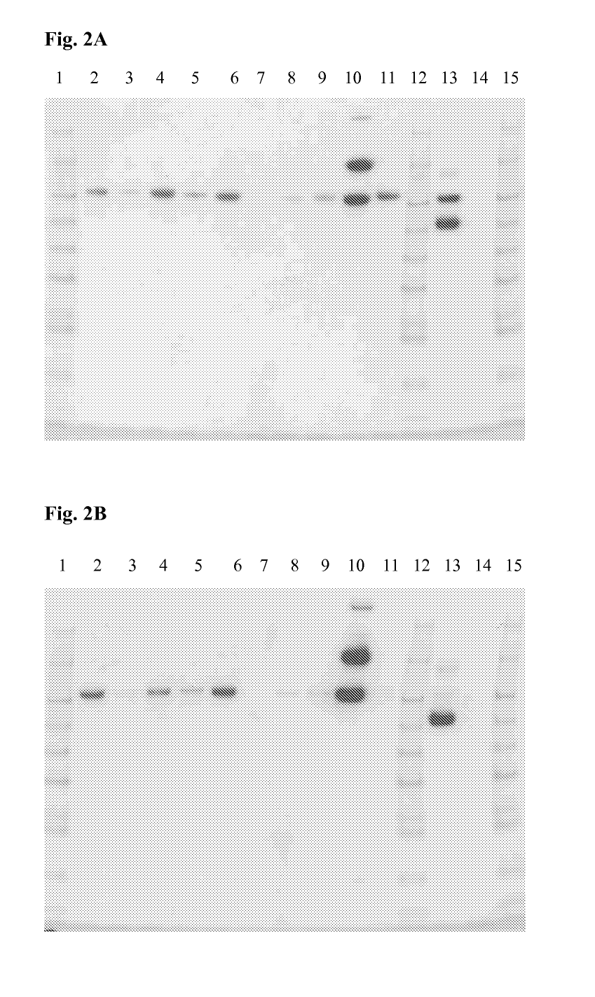

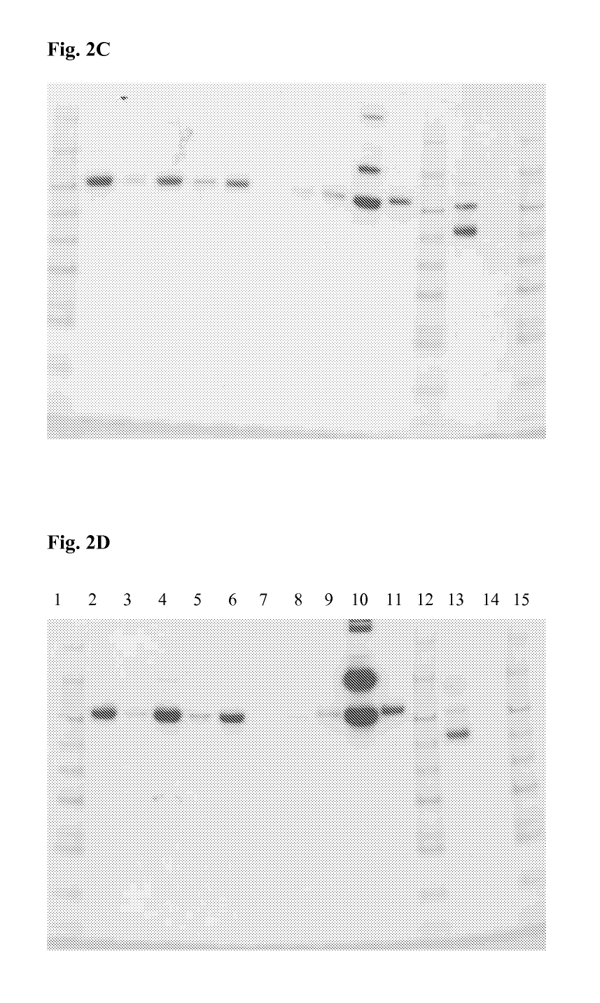

Immunoprecipitation (IP) with the human plasma samples used in the previous ELISA to evaluate whether similar results are obtained by different techniques (see FIG. 2A-D). There is a complete match between the ELISA and the IP results. This is very important because it is a qualitative validation of the results using two different techniques.

The following samples and antibodies were used in the IP experiments:

Samples:

| | Lane | | | | | | | | | | | | | | |
|---|---|---|---|---|---|---|---|---|---|---|---|---|---|---|---|
| | | | | | | | | | | | | | 13 | 14 | |
| | | | | | | | | | | | | | Positive | | |
| | | | | | | | | | | | | | and | | |
| | 1 | 2 | 3 | 4 | 5 | 6 | 7 | 8 | 9 | 10 | 11 | | negative | | |
| | | human plasma No | | | | | | | | | | 12 | control | | 15 |
| Sample | MWM | 1 | 2 | 3 | 4 | 5 | 6 | 7 | 9 | 8 | 10 | MWM | 11 | 12 | MWM |

MWM: Molecular weight marker; the controls were the same as in the ELISA assays.

MWM: Molecular weight marker; the controls were the same as in the ELISA assays.

Antibodies:

FIG. 2A: OLFM4-2/1

FIG. 2B: OLFM4-2/3

FIG. 2C: OLFM4-2/28

FIG. 2D: OLFM4-2/14

The results of the IP assays are given in FIG. 2A-D:

Positive samples in ELISA (#1-5 and #7-10) were also positive in IP. The negative sample in ELISA (#6) was also negative in IP. INS-1 OLFM4 supernatant and medium were used as positive and negative controls, respectively. This is a qualitative confirmation of the ELISA results by IP.

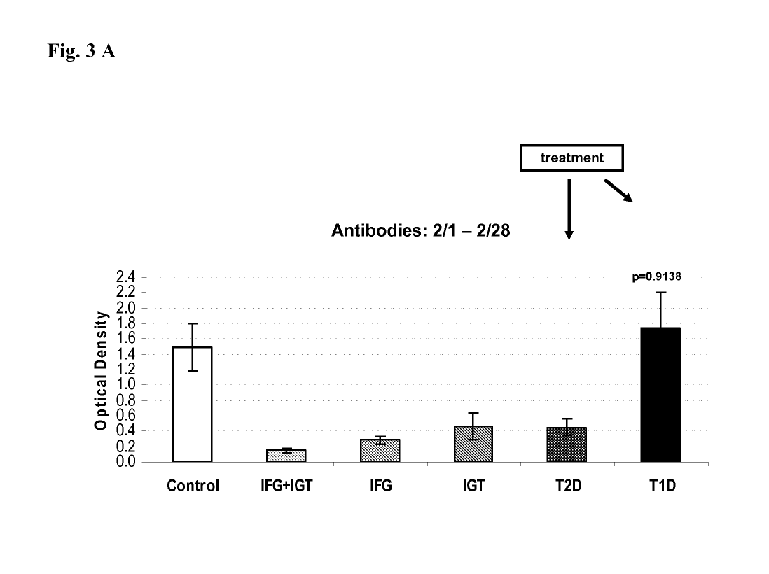

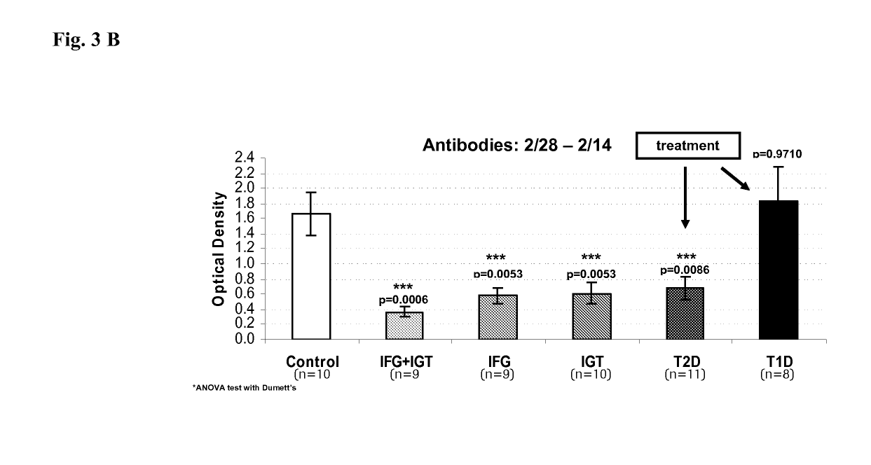

ELISA Results from Human Cross Sectional Cohort: OLFM4 Significantly Reduced in Pre-Diabetic and Diabetic Patients (Bratislava Cohort). FIG. 3A+3B

Human Plasma Cohort

Subjects Screening

About 200 subjects with metabolic risk of T2D from the register of the outpatient clinic fulfilling the following inclusion criteria:

Gender: Male

Age: 40-55

BMI: 25-32 kg/m2

HbA1C≦7.0%

underwent an oral glucose tolerance test (75 g). Exclusion criteria included previous knowledge of alterations in glucose metabolism, the use of drugs known to alter insulin secretion or action, and the presence of hepatic or endocrine diseases. Before the collection of the blood sample, height and weight were assessed using standard protocols. Body mass index (BMI) was calculated as weight in kilograms divided by height in meters squared. Whole-blood samples (20 ml) were collected into the EDTA test tubes from the antecubital vein after 10-12 h overnight fasting and also 2 h post-glucose load. To achieve an appropriate fasting state, precise instructions about the kind of food and time of their last intake were provided to participants. The plasma isolated by centrifugation was stored in 1 ml aliquots (10×) at −80° C. before the analysis.

Patients who signed an informed consent statement and who met the eligibility criteria were enrolled in the study. The Ethical committee of the Institute of Experimental Endocrinology of the Slovak Academy of Sciences approved the protocol of the study.

Diagnosis

Subjects were classified in 4 different groups, according to ADA guidelines 2005 (Diabetes Care. 2005 January; 28 Suppl 1:S37-42):

Healthy controls: Fasting Plasma Glucose (FPG)<5.6 mmol/l and Normal Glucose Tolerance (NGT)<7.8 mmol/l.

Impaired fasting glucose (IFG): IFG was defined by FPG value between ≧5.6 and <6.9 mmol/l and Normal Glucose Tolerance (NGT)<7.8 mmol/l at 2 hours post challenge.

Impaired glucose tolerance (IGT): IGT was defined by glucose concentration 2-hours post-load was between ≧7.8 and <11.1 mmol/l.

Impaired fasting glucose and glucose tolerance (IFG +IGT): IGT+IFG was defined by FPG value between ≧5.6 and <6.9 mmol/l and NGT value at 2 hourse post challenge between ≦7.8 and <11.1 mmol/l).

In addition a group of 8 diabetic patients with Type 1 and a group of 11 Type 2 patients—from the register of outpatient clinic was selected as well Patients with dysplipidemia were treated with hypolipidemic agents (e.g. statins or fibrates)

Summary Table of the Diabetic Subjects

| | T1DM | T2DM |
|---|---|---|
| Age | 25-50 | 40-55 |
| Gender | Male | Male |
| BMI (Body Mass Index) | 25-30 | 27-30 |
| Diesase duration (years) | 5-25 | 3-6 |
| HbA1c (%) | 7.0-9.0 | 7.0-9.0 |
| Antidiabetic Therapy | Insulin | Diet or Metformin or SU |

Average anthropometric and laboratory characteristics of subjects with type 1 (T1-DM), type 2 diabetes (T2-DM), impaired glucose tolerance (IGT), impaired fasting glucose (IFG), impaired glucose tolerance (IGT) and IFG+IGT

| | Controls | IFG | IGT | IFG + IGT | T2DM | T1DM |
|---|---|---|---|---|---|---|
| Number | 10 | 9 | 10 | 9 | 11 | 8 |
| Age | 46.7 ± 3.1 | 49. ± 2 | 50 ± 1 | 50 ± 1 | 50 ± 1 | 46 ± 2 |
| BMI | 24.1 ± 0.4 | 29.5 ± 0.7 | 30.2 ± 0.5 | 29.9 ± 0.8 | 30.1 ± 0.5 | 25.6 ± 0.4 |
| Duration | NA | NA | NA | NA | 3.9 | 18 ± 2 |
| of Diabetes | | | | | | |
| HbA1c | <7 | 6.2 ± 0.1 | 5.9 ± 0.2 | 5.9 ± 0.2 | 7.8 ± 0.2 | 7.4 ± 0.5 |
| Fasting | 5.1 ± 0.1 | 6.6 ± 0.1 | 5.6 ± 0.1 | 6.7 ± 0.1 | 9.8 ± 0.6 | 8.2 ± 0.6 |
| glucose | | | | | | |
| Post load | <7.8 | 6.4 ± 0.5 | 9.2 ± 0.3 | 10.2 ± 0.3 | NA | NA |
| glucose | | | | | | |
| Cholesterol | 4.7 ± 0.3 | 6.3 ± 0.4 | 5.6 ± 0.3 | 5.3 ± 0.3 | 5.3 ± 0.4 | 4.8 ± 0.3 |
| Triglycerides | 1.3 ± 0.2 | 3.2 ± 1.2 | 2.1 ± 0.3 | 2.7 ± 0.4 | 2.8 ± 0.7 | 0.8 ± 0.1 |

The following OLFM4 monoclonal antibody pairs were used in the ELISA assays:

FIG. 3A: Antibodies: 2/1-2/28

FIG. 3B: Antibodies: 2/28-2/14

ELISA results on a cross sectional cohort showed that OLFM4 levels are significantly lower in pre-diabetic patients (IFG+IGT, IFG, and IGT) than in healthy control patients (FIGS. 3A and 3B). The OLFM4 levels in T2DM patients are lower as well. Interestingly, OLFM4 levels in T1DM patients are higher although not significantly (ANOVA with Dunnett's correction). Both T2DM and T1DM groups of patients were under treatment.

Considering that OLFM4 is significantly reduced in untreated pre-diabetic patients, we claim that OLFM4 can be used as a marker for early T2D disease onset.

OLFM4 as Marker for Pancreatic β-Cells

Immunohistochemistry (IHC) staining of Human Tissue Array using the monoclonal antibody hOLFM4 1/46. The robust results showed no specific staining in any of the tissues tested whereas a very strong and specific signal was detected in β-cells of human islets (human pancreatic sections). Note that the pancreatic section in the tissue microarray appears negative because only exocrine tissue and no islet structure is present in the pancreatic spot of this tissue microarray.

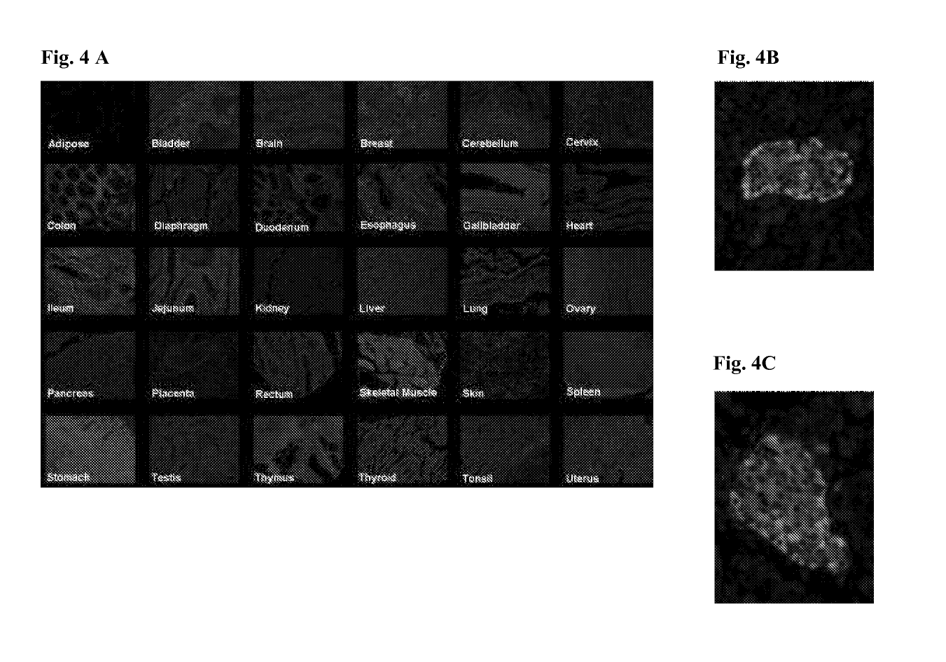

FIG. 4A: Human Tissue Array stained with monoclonal antibody hOLFM4 1/46

FIG. 4B and C: Human pancreatic islets stained with monoclonal antibody hOLFM4 1/46 OLFM4: green, glucagon: red, DAPI: blue).

Material and Methods

ELISA Protocol

Coating:

Coating-mAb: 5 m/ml in PBS 100 μl/well

→over night in humid box at 4° C.

→2× wash PBS-Tween

Blocking

B-Buffer

200 μl/well

→1 h at 37° C.

→2× wash PBS-Tween

Samples and Detection-mAb:

biotinylated detection-mAb, 1 μg/ml in B-Buffer: 25 μl/well

Samples (human plasma): dilution in B-Buffer, starting with undiluted plasma, in 1:2 steps to 1:128 (8 concentrations) 30 μl/well

add first 25 μl detection-mAb to the plates, then 30 μl of the samples

→over night in humid box on a shaker at 4° C.

→4× wash PBS-Tween

Conjugate:

PIERCE Streptavidine-HRPO (No 21126), 1 μg/ml in B-Buffer

50 μl/well

→1 h at room-T

→4× wash PBS-Tween

Substrate:

3,3′,5,5′-tetramethylbenzidine (TMB), 100 μl/well

stop the reaction after 5 min with 0.5M H₂SO₄, 100 μl/well

read at 450 nm

Cell Culture

Doxycline inducible rat insulinoma INS-1 hOLFM4 WT and INS-1 hOLFM4-His stable cell lines (expressing wild type (hOLFM4 WT) and His tagged (hOLFM4-His) human OLFM4 forms, respectively) were cultured as previously described (Wang et al. 2001). Both INS-1 cell lines were grown in RPMI 1640+GlutaMAX-1 medium (Invitrogen, Carlsbad, Calif.) containing 10 mM Hepes (pH 7.4), 1 mM sodium pyruvate, 50 μM 2-mercaptoethanol, 10% heat-inactivated fetal bovine serum (FBS), penicillin, and streptomycin. Fifty μg/ml G418 sulfate (Promega, Madison, WI) and 50 μg/ml zeosin (Invitrogen) were added for growth selection. Over-expression of hOLFM4 WT and hOLFM4-His was induced by 500 ng/ml doxycycline (Dox) (Sigma) for 96 hours. Cells were grown in a humidified incubator at 37° C. and 5% CO2 (subscript 2).

Immunoprecipitation (IP) and Immunoblotting (Western Blot, WB)

60-90% confluent cells were cultured with or without 500 ng/ml doxycycline for 96 hours in 10 cm petri dishes.Supernatants (cell culture media) were harvested in sterile conditions, centrifuged 10 minutes at 2000 rpm, and stored at 4° C. Cells were washed twice in 1× PBS and lysated with 1 mL lysis buffer. After 5 minutes, cells were collected in 1.5 mL Eppendorf tubes and centrifuged 5 minutes at full speed. Supernatants (whole cell extracts) were collected, aliquoted, snap frozen in liquid nitrogen and stored at −80° C. For IP, 3 mL of supernatant (cell culture media) were mixed with 1 μg of each mAb and incubated on an orbital-shaker 48 hours at 4° C. Twenty-five μL of Protein A Sepaharose CL-4B diluted 50% in 1× PBS-Tween (0.05%) were added to each reaction and incubated 1 hour at RT on an orbital-shaker. Tubes were spin-down and pellets were washed 2 times with 1× PBS-Tween (0.05%) and 1 time with 1× PBS. Thirty-five μL of 1× LDS-SB/10% β-ME were added to each pellet and the samples were vortex (what a word: vortexed??) vigorously and spun down before being loaded in a SDS-PAGE gel.

Immunoblotting, using enhanced chemiluminescence (Pierce, Rockford, Ill., USA) for detection, was performed as previously described (Wang H, J Biol Chem 2001).

Immunohistochemistry (IHC)

Formalin-fixed paraffin-embedded (FFPE) sections were used to assemble slides. Samples were dehydrated sequentially soaking the slides in xylol (×2), 100% EtOH, 95% EtOH, 80% EtOH, 70% EtOH, and 1× PBS (3 minutes each). Antigen retrieval was performed by soaking the slides in 1× citrate buffer and boiling them in a microwave (at 850 watts) for 3 minutes. After rinsing the slides twice with water, cells were permeabilized with 100 μL of 0.2% Triton in 1× PBS for 10 minutes at RT. After 3 washings with 1× PBS, blocking with 2% BSA in 1× PBS for 30′ to 1 h at RT was done. Three more washings with 1× PBS preceded the primary Ab incubation (1-2 hours at 37° C. or O/N at 4° C.). Three more washings with 1× PBS later, came the incubation with the secondary Ab for lh at RT in the dark. Three more washings and DAPI staining (5-10 minutes at RT in the dark). Three final washings and assembling of the cover slips.

FDA standard human tissue microarray (T8234700, Biochain) were stained with mouse anti-OLFM4 monoclonal antibody, followed by Alexa 488 conjugated donkey anti-mouse and Alexa 555 donkey anti-rabbit secondary antibodies (Invitrogene).

Human pancreatic sections obtained by Asterand were co-stained with both mouse anti-OLFM4 monoclonal antibody and rabbit anti-glucagon polyclonal antibody, followed by Alexa 488 conjugated donkey anti-mouse and Alexa 555 donkey anti-rabbit secondary antibodies (Invitrogene).

While there are shown and described presently preferred embodiments of the invention, it is to be distinctly understood that the invention is not limited thereto but may be otherwise variously embodied and practiced within the scope of the following claims.

1. A method for diagnosis of type II diabetes or determining the predisposition of an individual for developing type II diabetes comprising the steps of:
measuring in a tissue sample of the individual a level of Olfactomedin 4 (OLFM4) polypeptide, wherein an decreased level OLFM4 polypeptide in the sample of the individual compared to a level of OLFM4 polypeptide representative for a healthy population is indicative for type II diabetes or a predisposition for developing type II diabetes.

2. The method of claim 1, wherein the tissue is blood, preferably plasma.

3. A method for the identification of a compound for the treatment of type II diabetes comprising the steps of:
c) administering the compound to a non-human animal suffering from type II diabetes,
d) measuring in a tissue sample of the non-human animal of step a) a level of OLFM4 polypeptide, wherein an altered level of OLFM4 polypeptide in the tissue sample of the non-human animal of step a) compared to the level of OLFM4 polypeptide in a tissue sample of an non-human animal suffering from type II diabetes to which no compound has been administered is indicative for a compound for the treatment of type II diabetes.

4. The method of claim 3, wherein the tissue sample is blood, preferably plasma.

5. The method of claim 3 or 4, wherein the non-human animal is a rodent, preferably a mouse or rat.

6. The method of claim 5, wherein the rodent is a ZDF rat or an ob/ob mouse.

7. Use of OLFM4 polypeptide for the diagnosis of type II diabetes or for determining a predisposition of an individual for developing type II diabetes.

8. The use of claim 7, wherein the OLFM4 polypeptide is the human OLFM4 polypeptide.

9. Use of an antibody specifically binding to an OLFM4 polypeptide for the diagnosis of type II diabetes or for determining a predisposition of an individual for developing type II diabetes.

10. The use of claim 9, wherein the antibody binds to human OLFM4 polypeptide.

11. A kit for the diagnosis of type II diabetes or determining the predisposition for developing type II diabetes in an individual comprising:
d) an antibody specific for a OLFM4 polypeptide, preferably an antibody of claims 13-16,
e) a labeled antibody binding the antibody of a) or a labeled antibody binding the captured OLFM4 polypeptide of a) and
f) reagents for performing a diagnostic assay.

12. The kit of claim 11, wherein the specific antibody for the OLFM4 polypeptide binds the human OLFM4 polypeptide.

13. A monoclonal antibody directed to human OLFM4 polypeptide.

14. The antibody of claim 13, wherein the antibody comprises a CDR1 to CDR3 of a V_{H} domain of an antibody obtainable from a hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010) and a CDR1 to CDR3 of a V_{L} domain of an antibody obtainable from a hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010).

15. The antibody of claim 13 or 14, wherein the antibody comprises a V_{H} domain and a V_{L} domain of an antibody obtainable from a hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010).

16. The antibody of claims 13 to 15, wherein the antibody is produced by hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010).

17. A hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010).

18. A nucleic acid sequence comprising a sequence encoding a V_{H} domain of an antibody obtainable from a hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010).

19. A nucleic acid sequence comprising a sequence encoding a V_{L} domain of an antibody obtainable from a hybridoma cell line selected from the group consisting of OLFM4 2/3 (DSM ACC3012), OLFM4 1/46 (DSM ACC3011), OLFM4 2/1 (DSM ACC3013), OLFM4 2/14 (DSM ACC3014), OLFM4 2/28 (DSM ACC3015) and OLFM4 1/23 (DSM ACC3010).

20. Use of a OLFM4 polypeptide as a marker for β-cells of the pancreas.

21. A method for the detection of pancreatic β-cells in a tissue sample comprising:
c) providing a pancreatic tissue sample of an individual or a non-human animal,
d) detecting OLFM4 positive cells in the tissue sample of a), wherein the OLFM4 positive cells are β-cells.

22. The method of claim 21, wherein the OLFM4 positive cells are detected by an antibody specific for OLFM4, preferably an antibody of claims 13-16.

23. A kit for the detection of β-cells in a pancreatic tissue sample comprising:
a) an antibody specific for an OLFM4 polypeptide, preferably an antibody of claims 13-16,
b) a labeled antibody binding the antibody of a) and
c) reagents for performing an immunohistochemistry assay.

24. The methods and antibodies substantially as hereinbefore described, especially with reference to the foregoing examples.
